(19) United States
(12) Patent Application Publication
(10) Pub. No.: US 2021/0015783 A1
(43) Pub. Date: Jan. 21, 2021

(54) Pharmaceutical Compositions Containing Taxane-Cyclodextrin Complexes, Method of Making and Methods of Use

(71) Applicant: Meridian Lab, Buffalo Grove, IL (US)

(72) Inventors: William W. Zhao, Buffalo Grove, IL (US); John K. Thottathil, Buffalo Grove, IL (US); Xiaodong Sun, Buffalo Grove, IL (US); Xiangyu Dong, Buffalo Grove, IL (US)

(21) Appl. No.: 16/512,381

(22) Filed: Jul. 16, 2019

(51) Int. Cl. A61K 31/337; A61K 47/61; A61K 47/32; A61K 33/04; A61K 31/194; A61K 47/34

(52) U.S. Cl. CPC A61K 31/337; A61K 47/61; A61K 47/34; A61K 33/04; A61K 31/194; A61K 47/32

(57) ABSTRACT

Pharmaceutical formulations for parenteral administration comprising taxane, such as, cabazitaxel, compounds complexed with cyclodextrins and polyethylene glycol, methods of making the pharmaceutical formulations and methods of treating cancer patients using the pharmaceutical formulation.

### RELATED PATENT APPLICATIONS

This application is a continuation in part (CIP) application, which claims priority date of U.S. application Ser. No. 15/557,212, which was filed on Sep. 11, 2017, which, in turn, claims priority date of a PCT application No. PCT/US2016/022247, which was filed on Mar. 14, 2016, which, in turn, claims priority date of U.S. provisional application No. 62/133,698, which was filed on Mar. 16, 2015 and U.S. provisional application No. 62/304,543, which was filed on Mar. 7, 2016.

### TECHNICAL FIELD

This disclosure relates to pharmaceutical formulations of taxane compounds for parenteral administration, methods of making the pharmaceutical formulations and methods of treating cancer patients using the pharmaceutical formulations.

### BACKGROUND

Taxanes are diterpenes that are widely used in chemotherapy. They were originally discovered in plants of the genus Taxus (yews) and were first derived from these natural sources. Several are now chemically synthesized. Among the taxanes, the best known are paclitaxel (TAXOL) and docetaxel (TAXOTERE), which is a semisynthetic analog of paclitaxel. Taxanes exert their anti-cancer activity by inhibiting tubulin depolymerization in cells, thus inhibiting mitosis.

Parenteral administration by injection is the typical route of administration of taxanes. However, taxanes are substantially insoluble in water and in other commonly used medicinal parenteral organic solvents, which has presented a challenge to formulation for pharmaceutical use. Both Cabazitaxel and Docetaxel are typically formulated with POLYSORBATE 80 (Tween 80, a nonionic surfactant and emulsifier) to improve solubility. Paclitaxel is typically formulated with the nonionic surfactant CREMOPHOR EL (polyoxyethylated castor oil). Increased toxicity, including hypersensitivity reactions, anaphylaxis and other serious side effects are associated with these excipients. Pre-medication and additional treatment to prevent hypersensitivity is therefore often necessary. Such additional treatment includes hormone treatment, steroid medications, dexamethasone, diphenhydramine and cimetidine. In addition, the patient must be monitored closely for severe allergic reactions, such as observation of blood pressure, breathing rate, and heart rate. Increased toxicity and side effects sometimes restrict the continuation and completion of taxane treatments thus limiting its effectiveness.

The currently available pharmaceutical formulations of taxanes also suffer from unsatisfactory stability. For example, the docetaxel formulation has low stability and must be stored at or below room temperature protected from light. Alternatively, they need to be stored as a lyophilized solid prior to re-constitution to a liquid for patient administration. In the IV solution docetaxel has a tendency to precipitate and requires careful handling procedures such as to avoid shaking.

Formulations of docetaxel and paclitaxel requiring reduced amounts organic solvent and cyclodextrin have been reported. See U.S. Pat. Nos. 8,481,511 and 8,426,385. These formulations of either docetaxel or paclitaxel in complexes with hydroxypropyl-β-cyclodextrin (HP-β-CD) and/or sulfobutylether-β-cyclodextrin (SBE-β-CD) provide improved water-solubility and stability, as demonstrated by the Ka.

Although there has been some success in improving biocompatibility, in vivo tolerance, solubilization and formulation stability, there is still a need for further improvements in these parameters for pharmaceutical taxane compositions, particularly in parenteral formulations. The present invention addresses these needs. In contrast to the prior art cyclodextrin/taxane powders, the stable liquid pharmaceutical compositions comprising taxane/β-cyclodextrin-derivative complexes disclosed herein, based on accelerated aging studies, are expected to remain chemically and physically stable at temperatures from 0° C. to ambient temperature for at least two years, and may be diluted for use in either normal saline or dextrose. The present liquid pharmaceutical compositions also represent significant improvements over the prior art in that they do not contain toxic solubilizers such as CREMOPHOR EL and POLYSORBATE 80 (aka TWEEN 80).

Cabazitaxel (JEVTANAR) is one kind of taxanes. Cabazitaxel is, (1S,2S,3R,4S,7R,9S,10S,12R,15S)-4-(Acetyloxy)-15-(2R,35)-3-(tert-butoxy)carbonylamino-2-hydroxy-3-phenylpropanoyloxy)-1-hydroxy-9,12-dimethoxy-10,14,17,17-tetramethyl-11-oxo-6-oxatetracyclo 11.31″.07] heptadec-13-ene-2-yl benzoate. It is a microtubule inhibitor which is being investigated for multiple cancer treatments, such as head and neck cancer, non-Small cell lung cancer (NSCLC), Small cell lung cancer (SCLC), glioma, bladder cancer, gastric and esophageal cancer, breast cancer and ovarian cancer. Cabazitaxel has been approved under the brand of Jevtana for treating prostate cancer for patients with multidrug resistance (MDR) associated with paclitaxel and docetaxel and with many other anticancer drugs.

Due to its poor solubility in water, JEVTANAR used polysorbate 80 (Tween 80) as a solubilizing agent for its formulation which can cause hypersensitivity reaction and requires premedication with an antihistamine, a corticosteroid and an H antagonist.

Many attempts have been made to improve the cabazitaxel formulation by removing polysorbate 80 to improve the safety of the drug. Supratek claimed a formulation using SBECD without ethanol co-solvent.

While the prior art has proposed the formulation of docetaxel and cabazitaxel, no clinically viable liquid formulation without polysorbate 80 has been developed for fulfilling clinical use requirements which require long term shelf life stability for up to 24 months and after dilution stability for injection for minimal several hours (in excess of 6 hours).

### SUMMARY

In a first aspect the invention is directed to liquid pharmaceutical compositions comprising complexes of a taxane, such as cabazitaxel with a β-cyclodextrin derivative(a “taxane/β-cyclodextrin complex”), polyethylene glycol (PEG), and polyvinylpyrrolidone (aka povidone or PVP, for example povidone PF12 or PF17, an alcohol suitable for parenteral administration, and water. In certain embodiments, the liquid pharmaceutical compositions may comprise a selected amount by weight of the taxane, such as cabazitaxel, about 5 to about 200 parts by weight (p.b.w.) of the β-cyclodextrin or β-cyclodextrin derivative relative to the taxane, such as cabazitaxel, 10 to 50 p.b.w. of the PEG relative to the taxane, such as cabazitaxel, and about 5 to about 60 p.b.w. of the alcohol relative to the taxane, such as cabazitaxel. The remainder of the composition is water, such as pyrogen-free water suitable for injection. Stated on the basis of the weight ratios of the components of the liquid pharmaceutical compositions, the ratio of taxane, such as cabazitaxel to β-cyclodextrin or β-cyclodextrin derivative to PEG may be in the range of about 1:5:50 to about 1:100:10, with alcohol suitable for parenteral administration present in a weight ratio of 5 to 60 relative to taxane and water in a weight ratio of about 10 to 50 relative to cabazitaxel. In further specific embodiments of the foregoing liquid pharmaceutical compositions, the β-cyclodextrin may be present at about 40-100 p.b.w., the polyethylene glycol may be present at about 10-30 p.b.w., the alcohol may be present at about 5-about 40 p.b.w., and/or water may be present at about 10-about 50 p.b.w. relative to the weight of taxane, such as cabazitaxel. Specific examples include liquid pharmaceutical compositions comprising a weight ratio of taxane, such as cabazitaxel to β-cyclodextrin or β-cyclodextrin derivative to PEG of about 1:50:30, about 1:40:30, or about 1:60:20. Further specific examples include liquid pharmaceutical compositions comprising a weight ratio of taxane, such as cabazitaxel to β-cyclodextrin or β-cyclodextrin derivative to PEG to alcohol to water of about 1:50:30:20:10, about 1:40:30:30:20, or about 1:60:20:5:30.

In a further embodiment, the liquid pharmaceutical compositions may further comprise one or more of a weak organic acid, and an antioxidant. In a specific embodiment, the alcohol suitable for parenteral administration may be selected from the group consisting of alcohol, and combinations thereof; the weak organic acid may be citric acid, acetic acid, or phosphoric acid; the antioxidant may be selected from the group consisting of sodium bisulfite, sodium metabisulfite and combinations thereof. In a further specific embodiment, the liquid pharmaceutical composition may comprise about 2-about 5 p.b.w. of the weak organic acid (to adjust the final pH as desired), about 0.01-about 0.1 p.b.w. of the antioxidant, ethanol, benzylalcohol, and combinations thereof; the weak organic acid may be citric acid, acetic acid, or phosphoric acid; the antioxidant may be selected from the group consisting of sodium bisulfite, sodium metabisulfite and combinations thereof. In a further specific embodiment, the liquid pharmaceutical composition may comprise about 2-about 5 p.b.w. of the weak organic acid (to adjust the final pH as desired), about 0.01-about 0.1 p.b.w. of the antioxidant.

In any of the foregoing embodiments of the liquid pharmaceutical compositions, the taxane may be paclitaxel, docetaxel, cabazitaxel, ortataxel, tesetaxel or a combination thereof. In any of the foregoing embodiments of the liquid pharmaceutical formulation, the β-cyclodextrin may be selected from the group consisting of sulfobutylether-β-cyclodextrin, hydroxypropyl-sulfobutylether-β-cyclodextrin, and combinations thereof. In any of the foregoing embodiments, the alcohol suitable for parenteral administration may be selected from the group consisting of ethanol, benzyl alcohol, and combinations thereof. In any of the foregoing embodiments, the polyethylene glycol may be a low molecular weight PEG, and in specific embodiments may be selected from the group consisting of PEG 200, PEG 300, PEG 400, PEG 600, and combinations thereof. The selected soluble povidone is preferably suitable for parenteral use, i.e., with low endotoxin levels. Examples include Kollidon® soluble povidones from BASF, such as Kollidon® PF12 (M.sub.w 2,000-3,000), Kollidon® PF17 (M.sub.w 7,000-11,000), and mixtures thereof.

In a second aspect, the invention provides methods of making the liquid pharmaceutical compositions described above. The methods may comprise the steps of:
a) dissolving a taxane, such as cabazitaxel, in an alcohol suitable for parenteral administration to form a taxane, such as cabazitaxel solution;
b) dispersing a low molecular weight polyethylene glycol and PVP (povidone) in water to form a dispersion, and dissolving the β-cyclodextrin derivative in the dispersion;
c) combining the taxane, such as cabazitaxel solution obtained in step (a) and the dispersion obtained in step (b);
d) adjusting the combination obtained in step (c) to a final volume with the alcohol or water; and
e) optionally, adjusting pH of the final volume by addition of a weak organic acid.

In a specific embodiment, the method of making the liquid pharmaceutical compositions described above comprises:
a) combining the taxane, such as cabazitaxel with a portion of the alcohol suitable for parenteral administration and mixing until the taxane, such as cabazitaxel, is dissolved;
b) dispersing the polyethylene glycol and optionally PVP (povidone) in the water;
c) adding the β-cyclodextrin derivative, or mixture thereof, to the polyethylene glycol/water dispersion obtained in step (b), and mixing until the β-cyclodextrin derivative is dissolved;
d) adding a remaining portion of the ethanol to the mixture obtained in step(c) and mixing to form a homogenous solution; and
e) adding the taxane/alcohol, such as cabazitaxel/alcohol mixture obtained in step (a) to the solution obtained in step (d) while mixing under an inert atmosphere to obtain a homogeneous solution of the liquid pharmaceutical composition.

Certain embodiments of the methods of making the pharmaceutical compositions may include a further step of sterilizing the homogeneous solution obtained. The sterilization may be filter sterilization. In further embodiments, in step (a), the taxane, such as cabazitaxel, may be combined with about ¼ to about ½ of the total amount of alcohol suitable for parenteral administration in the final formulation. In other embodiments, the method may further comprise the step of mixing one or more of a weak organic acid, and an antioxidant in the water in step (b). In some embodiments, the steps (c), (d) (e) of the process may be carried out under an inert atmosphere such as nitrogen. In a modification of such embodiments, all steps of the process are carried out under an inert atmosphere such as nitrogen. In certain embodiments, the compounds and their amounts used in the methods of making the liquid pharmaceutical compositions may be those discussed above with respect to the liquid pharmaceutical composition, per se.

In a third aspect, the invention provides methods of treating cancer in a patient by parenteral administration to the cancer patient of any of the foregoing embodiments of the liquid pharmaceutical compositions, in an amount sufficient to treat the cancer. In certain embodiments, the liquid pharmaceutical formulation may further comprise β-cyclodextrin derivative. wherein the β-cyclodextrin derivative is selected from the group consisting of sulfobutylether-β-cyclodextrin sodium, hydroxypropyl-sulfobutylether-β-cyclodextrin, and combination thereof. In further embodiment, the liquid pharmaceutical formulation further comprises povidone. In certain embodiments, a selected amount of the cabazitaxel by weight is about 5 to about 200 parts by weight (p.b.w.) of the β-cyclodextrin derivative relative to the cabazitaxel. Accordingly, the invention also provides use of the liquid pharmaceutical formulation of any of the foregoing embodiments of the liquid pharmaceutical compositions for the treatment of cancer.

### BRIEF DESCRIPTION OF THE DRAWINGS

The Fig. shows the results of accelerated aging stability studies for an exemplary liquid pharmaceutical composition as described herein.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein the term “taxane” refers, individually and collectively, to diterpene compounds of the taxane family. Included are naturally occurring taxanes, derivatives of such naturally occurring taxanes, semi-synthetic taxanes and synthetic taxanes.

As used herein the term “taxane/β-cyclodextrin complex” refers to the complex formed by the interaction of a β-cyclodextrin or a derivative of a β-cyclodextrin molecule having a 7-membered sugar ring (β-cyclodextrin ring)with a hydrophobic diterpene which is a member of the taxane drug family (including naturally occurring compounds and their derivatives, semi-synthetic compounds, and synthetic compounds) in a manner which improves the solubility of the taxane in aqueous media.

As used herein, the term “physical stability” refers to an absence of precipitate formation, turbidity, opaqueness, or coagulation/gelatinous formation in the liquid pharmaceutical taxane/β-cyclodextrin complex compositions overtime.

As used herein, the term “chemical stability” refers to an absence of chemical impurity formation and/or an absence of taxane degradation in the liquid pharmaceutical taxane/β-cyclodextrin complex compositions over time.

As used herein the term “parenteral administration” refers to administration of a composition to a patient via injection or infusion. Included are intravenous injection, subcutaneous injection and intramuscular injection. Also included is infusion, which is typically administration via the intravenous route over a period of time. “Suitable for parenteral administration” refers to acceptable safety of a component when parenterally administered, which is typically established by regulatory approval of the component for such use.

As used herein the term “treatment” of cancer and its equivalents refers to reducing the symptoms or extent of cancer in a patient by administration of a drug or drug formulation. Treatment may also include curing the cancer or reducing the cancer burden to levels below detection limits or preventing its recurrence.

As used herein the term “about,” refers to including 20% range of a value. For example. About value A means that the value is equal within A−20% A and A+20% A.

In certain embodiments, the liquid pharmaceutical compositions described herein comprise taxane complexes with a β-cyclodextrin or β-cyclodextrin derivative, polyethylene glycol (PEG), polyvinylpyrrolidone (aka povidone or PVP, for example povidone PF12 or PF17, an alcohol suitable for parenteral administration, and water. The taxane portion of the taxane/β-cyclodextrin complex may be any naturally-occurring, semi-synthetic or synthetic hydrophobic taxane known in the art, including those that have regulatory approval for clinical use (e.g., paclitaxel, docetaxel, and cabazitaxel,) as well as those that are in development or otherwise known in the art. Examples of suitable taxanes, such as cabazitaxel, that are of scientific interest but are not currently approved for commercial use include DJ-927(tesetaxel), ortataxel, XRP9881, DHA-paclitaxel, and BMS-184476). In specific embodiments, the liquid pharmaceutical compositions described herein comprise paclitaxel, docetaxel, cabazitaxel or a combination thereof, in the taxane/β-cyclodextrin-derivative complex.

The β-cyclodextrin portion of the taxane/β-cyclodextrin complex may be any suitable derivative of β-cyclodextrin. Specific examples include sulfobutylether-β-cyclodextrin (SBE-β-CD) (which may or may not be as its sodium salt), hydroxypropyl-sulfobutylether-β-cyclodextrin (HP-SBE-β-CD), In specific embodiments, the liquid pharmaceutical compositions described herein comprise sulfobutylether-β-cyclodextrin sodium, hydroxypropyl-sulfobutylether-β-cyclodextrin or a combination thereof, in the taxane/β-cyclodextrin complex.

The polyethylene glycol component of the liquid pharmaceutical compositions described herein may be any pharmaceutically acceptable PEGs known in the art that are suitable for pharmaceutical applications and that are suitable to provide improved physical and chemical stability to the composition. In certain embodiments, the PEG component of the compositions is a low molecular weight PEG which has a molecular weight of 2000 Daltons or less. In a particular embodiment, the PEG component of the composition has a molecular weight of from about 200 Daltons to about 800 Daltons. In a specific embodiment, the PEG component of the composition has a molecular weight of 300 Daltons, 400 Daltons, or 600 Daltons.

The alcohol suitable for parenteral administration used in the liquid pharmaceutical compositions described herein may be any alcohol that is suitably safe for parenteral administration (whether diluted with water or undiluted). Typically, the alcohol will be approved by regulatory authorities for parenteral use. Examples of suitable pharmaceutically acceptable alcohols include ethanol, benzyl alcohol, and combinations thereof. In a specific embodiment, the alcohol component of the liquid pharmaceutical compositions described herein is ethanol.

The taxane/β-cyclodextrin complex, low molecular weight PEG, and alcohol suitable for parenteral administration are the basic components of the liquid pharmaceutical compositions. Typically, the remainder of the composition is water, for example water for injection.

In certain embodiments, the pH of the liquid pharmaceutical composition may optionally be adjusted by inclusion of a weak organic acid in an amount sufficient to further improve the stability of the taxane. Taxanes typically show improved stability in the acidic pH range. If necessary or desired, the pH may be adjusted into the range of about 3-8, or 4-7, or 3.5-4 by addition of the weak organic acid. The weak organic acid added to adjust the pH may be, for example, citric acid, acetic acid, ascorbic acid, aspartic acid, formic acid, lactic acid, glutamic acid, phosphoric acid or succinic acid. However, suitable compositions in the range of pH 3.5-4 can often be obtained without adjustment of the pH with weak organic acid. The liquid pharmaceutical compositions described herein may also optionally contain one or more antioxidants, as is known in the art. Any natural or synthetic antioxidant that is suitable for parenteral administration may be included in the compositions. Examples of suitable antioxidants include sodium bisulfite, sodium metabisulfite, and combinations thereof. A specific embodiment of the liquid pharmaceutical compositions described above is a composition which comprises the base components (i.e., taxane/β-cyclodextrin complex, such as cabazitaxel/β-cyclodextrin, low molecular weight polyethylene glycol, alcohol suitable for parenteral administration, and water) as follows: a selected amount of the taxane by weight, about 5 to about 200 p.b.w. of the β-cyclodextrin or β-cyclodextrin derivative with respect to the taxane, about 50 to about 20 p.b.w. of the polyethylene glycol relative to the taxane, about 5 to about 60 p.b.w. of the alcohol suitable for parenteral administration relative to the taxane, such as cabazitaxel, and about 10 to about 50 p.b.w. of the water relative to the taxane, such as cabazitaxel. Any or all of the optional components of the compositions may be added to this base formulation in the amounts disclosed herein. A further specific embodiment of the liquid pharmaceutical compositions comprise a selected amount of the taxane, such as cabazitaxel by weight, about 5 to about 100 p.b.w. of the β-cyclodextrin or β-cyclodextrin derivative with respect to the taxane, such as cabazitaxel, about 50 to about 10 p.b.w. of the polyethylene glycol relative to the taxane, such as cabazitaxel, about 5 to about 60 p.b.w. of the alcohol suitable for parenteral administration relative to the taxane, such as cabazitaxel, and about 10 to about 50 p.b.w. of the water relative to the taxane, such as cabazitaxel. As a specific example, the liquid pharmaceutical compositions may comprise 1 p.b.w. taxane, such as cabazitaxel, about 40 to about 60 p.b.w. of the β-cyclodextrin or β-cyclodextrin derivative relative to the taxane, such as cabazitaxel, about 20 to about 30 p.b.w. of the polyethylene glycol relative to the taxane, such as cabazitaxel, about 5 to about 30 p.b.w. of the alcohol suitable for parenteral administration relative to the taxane, such as cabazitaxel, and about 10 to about 30 p.b.w.

water relative to the taxane, such as cabazitaxel.

Weight ratios of the components of certain specific embodiments of the liquid pharmaceutical compositions include about 1:40:30, about 1:50:30 or about 1:60:20 (taxane: β-cyclodextrin derivative: PEG). Such embodiments include; for example, weight ratios of about 1:10:30:20:10 (taxane, such as cabazitaxel: β-cyclodextrin derivative:PEG:alcohol:water), about 1:20:30:20:10 (taxane, such as cabazitaxel: β-cyclodextrin derivative:PEG:alcohol:water), about 1:40:30:30:20 (taxane, such as cabazitaxel:β-cyclodextrin derivative:PEG:alcohol:water), about 1:50:30:20:10 (taxane, such as cabazitaxel: β-cyclodextrin derivative:PEG:alcohol:water), about 1:60:20:5:30 (taxane, such as cabazitaxel:β-cyclodextrin derivative:PEG:alcohol:water), and about 1:70:20:40:40 (taxane, such as cabazitaxel:β-cyclodextrin derivative:PEG:alcohol:water), about 1:80:20:40:40 (taxane, such as cabazitaxel: β-cyclodextrin derivative:PEG:alcohol:water) or about 1:100:10:10:50 (taxane, such as cabazitaxel:β-cyclodextrin derivative:PEG:alcohol:water).

A further specific embodiment of any of the foregoing liquid pharmaceutical compositions is a composition which further comprises optional components as follows: about 2-about 5 p.b.w. of a weak organic acid relative to the taxane, such as cabazitaxel, (to adjust the final pH), about 0.01-about 0.1 p.b.w. of an antioxidant relative to the taxane, such as cabazitaxel.

In a further embodiment, any of the foregoing liquid pharmaceutical compositions may optionally further comprise a soluble polyvinylpyrrolidone (aka povidone or PVP, for example povidone PF12 or PF17). The inclusion of povidone in the composition further improves its chemical and physical stability. The selected soluble povidone is preferably suitable for parenteral use, i.e., with low endotoxin levels. Examples include Kollidon® soluble povidones from BASF, such as Kollidon® PF12 (M.sub.w 2,000-3,000), Kollidon® PF17 (M.sub.w 7,000-11,000), and mixtures thereof. Other suitable low-endotoxin soluble povidones are also available commercially, including Plasdone™ C-12 (nomina M.sub.w 4,000) and Plasdone™ C-17 (nominal M.sub.w 10,000) from Ashland. Because it is difficult to determine the molecular weight of povidone polymers directly, the K-value has been adopted to classify the various molecular weights of povidones. The K-value is a function of the average degree of polymerization and the intrinsic viscosity of the polymer and is calculated from the kinematic viscosity of an aqueous polymer solution. Examples of povidones for use in the invention, characterized by their K-value, include povidones having K-values of 10.2-13.8, povidones having K-values of about 15.5-about 17.5, and mixtures thereof. Specific examples include povidones having K-values of about 12, povidones having K-values of about 17, and mixtures thereof.

In some embodiments the liquid pharmaceutical compositions comprise a weight ratio of taxane to povidone of about 1:1 to about 1:10. In a specific embodiment the weight ratio of taxane to povidone is about 1:5. Specific examples include liquid pharmaceutical compositions comprising a weight ratio of taxane to β-cyclodextrin derivative to PEG to povidone of about 1:50:30:5, about 1:40:30:5, or about 1:60:20:5. Further specific examples include liquid pharmaceutical compositions comprising a weight ratio of taxane to β-cyclodextrin derivative to PEG to povidone of about 1:50:20:5 and about 1:60:15:5. Other specific examples include liquid pharmaceutical compositions comprising a weight ratio of taxane, such as cabazitaxel, to β-cyclodextrin derivative to PEG to povidone of about 1:40:20:5 and about 1:30:30:5.

The liquid pharmaceutical compositions described herein are physically and chemically stable as liquid solutions for an extended period of time. That is, under conditions of accelerated aging the impurities remain low (i.e., below allowable limits set by the United States Pharmacopeia USP35 standards for taxanes) and the taxane shows no decomposition, precipitation or turbidity formation. The compositions therefore provide the advantage of long-term stable storage in a liquid form that reduces risk to medical personnel handling the compositions. To use the liquid pharmaceutical taxane/β-cyclodextrin compositions for treatment of a cancer patient, the concentrated composition as described above is diluted to the appropriate dose in a pharmaceutically acceptable aqueous medium (for example, in an IV or infusion bag at the patient bed-side) and delivered parenterally to the patient for treatment of the cancer. The reconstitution and mixing step that increases exposure of medical personnel when using lyophilized powder formulations is therefore eliminated. Also eliminated are problems associated with incomplete dissolution, precipitation after dissolution and frothing during dissolution, with the avoidance of very sensitive shaking/mixing restrictions required for prior art taxane compositions).Persons skilled in the art can determine the effective dose and administration protocol to achieve treatment of specific cancers using the liquid pharmaceutical compositions described herein. Accordingly, use of the liquid pharmaceutical compositions described herein for treatment of cancer is an embodiment of the invention. Generally, the route of delivery of the diluted composition will be by intravenous injection or intravenous infusion.

The present liquid pharmaceutical compositions also have the advantage that they can be diluted in either saline or dextrose. This is in contrast to the lyophilized powders disclosed in U.S. Pat. Nos. 8,481,511 and 8,426,385, which form stable solutions only when reconstituted in dextrose, i.e., reconstitution in saline results in precipitation of the taxane. Because the liquid pharmaceutical compositions described herein can be diluted directly into an infusion bag and do not contain toxic solubilizers and emulsifiers, treatment of patients using the liquid pharmaceutical compositions substantially reduces the risk of toxicity to both medical personnel (who must handle the compositions) and to patients (who are at risk for potentially life-threatening hypersensitivity reactions to solubilizers used in the prior art).

In one aspect, the liquid pharmaceutical compositions disclosed herein may be used in methods for treating cancer patients by administering any of the foregoing liquid pharmaceutical formulations to a cancer patient in an amount sufficient for cancer treatment. Accordingly, it is also to be understood that embodiment of such methods or treatment include use of any of the foregoing liquid pharmaceutical formulations for treatment of cancer. In general, the amount of taxane, such as cabazitaxel, administered and the duration of treatment are within the skill and knowledge of the medical practitioner. However, it is to be understood that the present taxane, such as cabazitaxel, formulations do not need to be reconstituted prior to administration. That is, the present liquid pharmaceutical taxane formulations can simply be diluted using saline or dextrose to obtain the desired taxane concentration for administration to the patient, including dilution by direct injection of the concentrate into the patient's IV bag.

The liquid pharmaceutical compositions described herein may be prepared by the following general method:
a) dissolving a taxane, such as cabazitaxel, in an alcohol suitable for parenteral administration to form a taxane solution;
b) dispersing a low molecular weight polyethylene glycol and/or PVP in water to form a dispersion, and dissolving a β-cyclodextrin or β-cyclodextrin derivative in the dispersion;
c) combining the taxane such as cabazitaxel, solution obtained in step (A) and the dispersion obtained in step (b) to form a homogeneous solution; and
d) if needed, adjusting the homogeneous solution obtained in step (c) to a final volume with the alcohol or water.

The liquid pharmaceutical compositions described herein may also be prepared by the following specific method:
a) combining the taxane, such as cabazitaxel, with a portion of the alcohol and mixing until the taxane is dissolved;
b) dispersing the polyethylene glycol and/or PVP in the water;
c) adding the β-cyclodextrin derivative, or mixture thereof, to the polyethylene glycol/PVP/water dispersion obtained in step (b), and mixing until the β-cyclodextrin is dissolved;
d) adding a remaining portion of the alcohol to the mixture obtained in step
(c) and mixing to form a solution; and
e) adding the taxane/alcohol, such as cabazitaxel/alcohol mixture obtained in step (a) to the solution obtained in step (d) while mixing to obtain a homogeneous solution of the liquid pharmaceutical composition.

In certain embodiments, steps (c), (d) and (e) of the above method may be conducted under an inert atmosphere, for example nitrogen or argon. In alternative embodiments all of the steps of the preparation methods may be conducted under an inert atmosphere, for example nitrogen or argon.

The portion of the alcohol suitable for parenteral administration that is added in step (a) in some embodiments may be about ¼ to about ½ of the total amount in the final composition. The remaining amount of alcohol may then be added in step (d). The ratio of each component used in the method is calculated based on the desired composition of the final product, as discussed above and the amount of each component used in the method is calculated based on the desired final amount of the final product, as discussed above. Any or all of the steps of the process may be carried out under an inert atmosphere such as nitrogen or argon. In contrast to the prior art lyophilized powder formulations discussed above, the alcohol suitable for parenteral administration is not removed in the manufacturing process and is a component of the final liquid pharmaceutical compositions described herein.

If any or all of the optional components of the composition (weak organic acid, and antioxidant) are to be included in the liquid pharmaceutical composition, they may be added to the water in step (b) prior to addition of the polyethylene glycol and PVP and mixed to dissolve. The PEG may then be dispersed in the solution of water and optional component(s). The weak organic acid may also be added at to the homogeneous solution at the end of the process to adjust the final pH to the desired value.

In certain embodiments, the water used for preparation of the liquid pharmaceutical compositions may be water for injection (WFI), which is pyrogen-free. It may still be desirable, however, to sterilize the final product before dispensing it into individual vials and sealing. Sterilization may be accomplished by filter sterilization, for example by filtration through a 0.22 μm membrane filter.

### EXAMPLES

### Accelerated Aging Stability Studies

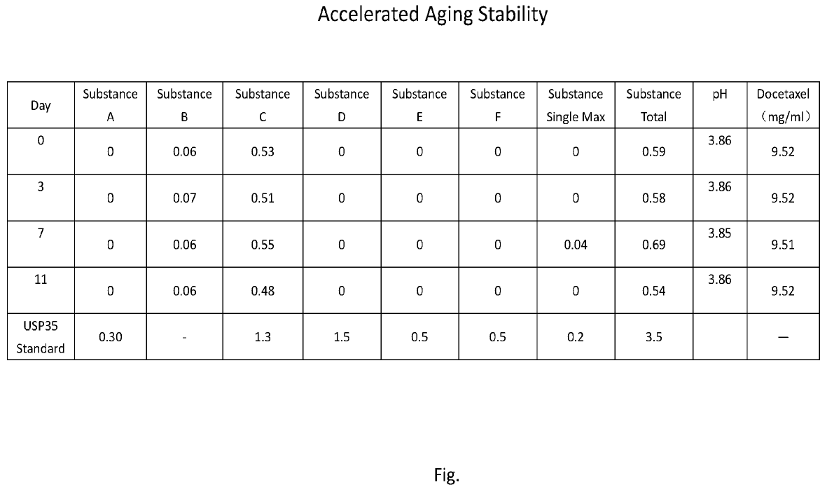

A liquid pharmaceutical composition as described above, comprising ˜10 mg/mL docetaxel was diluted and subjected to accelerated aging at 40° C. for eleven days. The levels of impurities as specified by USP35 (United States Pharmacopeia 35) for taxane compositions were analyzed by HPLC at regular intervals. Stability was tested at 6 hours on day 0. Impurity levels in the liquid pharmaceutical compositions were compared to the allowable level according to the USP35 standard. The results are shown in FIG. 1. It can be seen that all impurities (Substances A-F) are substantially below the USP35 limits for the entire 1-day period of accelerated aging. In addition, the impurities that are present do not increase over the 11 days of testing, and no new impurities or degradation products were formed indicating that the composition is chemically stable. The composition was also physically stable, as no precipitate or turbidity was formed.

In addition, the concentration of docetaxel remained constant at about 9.52 mg/mL throughout the 11 days of accelerated aging. This confirms that the taxane does not decompose even at about 40° C. for 11 days.

### Stability of Exemplary Formulations

Various liquid pharmaceutical compositions were prepared as described above, comprising weight ratios of docetaxel to cyclodextrin to PEG from about 1:5:50 to about 1:100:10. pH of the formulations was about 3.5-4.0 without adjustment. Stability at about 40° C. with respect to the level of impurities was analyzed by HPLC at regular intervals as specified by USP35 (United States Pharmacopeia 35) for taxane compositions. Impurity levels in the liquid pharmaceutical compositions were compared to the allowable level according to the USP35 standard. The time point at which the levels of impurities exceeded the allowable level for each of the compositions is shown in Table 1:

TABLE 1
Weight Ratios of drug to excipients

| | | | | | | 40° C. Stability | Diluted Stability | |
|---|---|---|---|---|---|---|---|---|
| Example | Docetaxel | cyclodextrin | PEG300 | ethanol | water | (Undiluted) | (Ambient) | |
| 1 | 1 | 100 | 10 | 10 | 50 | 6 mo. | >=6 | h |
| 2 | 1 | 80 | 20 | 40 | 40 | 6 mo. | >=6 | h |
| 3 | 1 | 60 | 20 | 5 | 30 | 6 mo. | >=6 | h |
| 4 | 1 | 50 | 30 | 20 | 10 | 6 mo. | 6 | h |
| 5 | 1 | 40 | 30 | 30 | 20 | 6 mo. | 5 | h |
| 6 | 1 | 30 | 40 | 40 | 15 | 5 mo. | 4 | h |
| 7 | 1 | 20 | 50 | 40 | 15 | 5 mo. | 2 | h |
| 8 | 1 | 10 | 50 | 50 | 15 | 4 mo. | 1 | h |
| 9 | 1 | 5 | 50 | 60 | 10 | 2 mo. | 0.5 | h |

Physical stability of the samples was confirmed visually, and no turbidity, cloudiness or precipitation was observed during the time periods in which the samples were chemically stable. Formulations comprising at least about 1:5:50 parts by weight of cabazitaxel: cyclodextrin: PEG300 exhibited extended chemical stability on accelerated aging (2 months). Chemical stability could be increased to at least six months by increasing the proportion of cyclodextrin derivative and reducing the proportion of PEG300 (formulations having weight ratios from about 1:40:30 to about 1:100:10). Intermediate duration of chemical stability was obtained with ratios from about 1:10:50 to about 1:30:40. Table 2 shows the formulations tested in Table 1 normalized to a single vial containing 20 mg. of docetaxel:

TABLE 2
Normalized to single vial (20 mg)

| Example | Docetaxel | cyclodextrin | | PEG 300 | | ethanol | | water | | 40° C. Stability | Diluted stability | |
|---|---|---|---|---|---|---|---|---|---|---|---|---|
| 1 | 20 mg | 2 | g | 0.2 | g | 0.2 | g | 1 | g | 6 mo. | >=6 | h |
| 2 | 20 mg | 1.6 | g | 0.4 | g | 0.8 | g | 0.8 | g | 6 mo. | >=6 | h |
| 3 | 20 mg | 1.2 | g | 0.4 | g | 0.1 | g | 0.6 | g | 6 mo. | >=6 | h |
| 4 | 20 mg | 1 | g | 0.6 | g | 0.4 | g | 0.2 | g | 6 mo. | 6 | h |
| 5 | 20 mg | 0.8 | g | 0.6 | g | 0.6 | g | 0.4 | g | 6 mo. | 5 | h |
| 6 | 20 mg | 0.6 | g | 0.8 | g | 0.8 | g | 0.3 | g | 5 mo. | 4 | h |
| 7 | 20 mg | 0.4 | g | 1 | g | 0.8 | g | 0.3 | g | 5 mo. | 2 | h |
| 8 | 20 mg | 0.2 | g | 1 | g | 1 | g | 0.3 | g | 4 mo. | 1 | h |
| 9 | 20 mg | 0.1 | g | 1 | g | 1.2 | g | 1 | g | 2 mo. | 0.5 | h |

In this invention, we have developed a formulation of cabazitaxel that is free of polysorbate 80 in a liquid formulation that is stable in storage and in dilution bag for injection that satisfy the clinical needs.

The formulation compromises of cabazitaxel, cyclodextrin of SBECD or HP-SBECD, ethanol, PEG or similar actant and PVP and water. The ratio of cabazitaxel and cyclodextrin derivative is about 1:10-about 1:200 in weight.

### Pharmaceutical Compositions Containing Povidone

Pharmaceutical compositions were prepared as above with the addition of povidone PF12. Compositions comprised docetaxel:ethanol:PEG300:cyclodextrin:povidone in weight ratios as shown in Table 3.

TABLE 3

| Example | Docetaxel | Povidone PF12 | cyclodextrin | PEG 300 | ethanol | Stability |
|---|---|---|---|---|---|---|
| M | 1 | 5 | 45 | 0 | 18 | API solidified during stirring |
| N | 1 | 5 | 60 | 15 | 30 | Completely dissolved after 30 min. Reconstitute |
| O | 1 | 5 | 50 | 20 | 24 | Completely dissolved after 30 min. Reconstitute |
| | | | | | | Acceptable at 25° C. after 6 months |

### Pharmaceutical Compositions Containing Cabazitaxel

Pharmaceutical compositions were prepared as above with the addition of cabazitaxel.

TABLE 4

| | | | | | | | |
|---|---|---|---|---|---|---|---|
| Formulation # | 1 | 2 | 3 | 4 | 5 | 6 | 7 |

Formulation composition (based on mg/ml)

| | | | | | | | |
|---|---|---|---|---|---|---|---|
| API | 10 | 10 | 10 | 10 | 10 | 10 | 10 |
| Ethanol | 0 | 0 | 0 | 200 | 300 | 350 | 200 |
| PEG 300 | 0 | 0 | 0 | 0 | 0 | 0 | 150 |
| SBECD | 500 | 500 | 500 | 500 | 500 | 500 | 500 |
| Water for Inj. | ~40 ml | ~40 ml | ~80 ml | ~80 ml | 80 ml | ~300 | ~250 |
| Observation | API not | API not | API not | API not | SBECD not | API soluble | API soluble, |
| of the | completely | completely | completely | completely | completely | | but the |
| formulation | soluble, | soluble, | soluble, | soluble, | soluble, | | solution |
| | solution | solution | solution | solution | solution | | viscosity |
| | turbidity | turbidity | turbidity | turbidity | turbidity | | is high |
| Storage | NA | NA | NA | NA | NA | Stratification | No API |
| stability | | | | | | occurred | precipitation |
| (25° C.) | | | | | | after one month | after 12 months |
| Dilution | NA | NA | NA | NA | NA | 3 hours | 8 hours |
| stability time | | | | | | | |
| (observation of | | | | | | | |
| API precipitation) | | | | | | | |
| Results | | | | | | The large | |
| explanation | | | | | | quantity of | |
| | | | | | | ethanol helped | |
| | | | | | | to increase | |
| | | | | | | the API | |
| | | | | | | solubility | |

| Formulation # | 8 | 9 | 10 | 11 |
|---|---|---|---|---|
| Formulation composition (based on mg/ml) | | | | |
| API | 10 | 10 | 10 | 10 |
| Ethanol | 200 | 200 | 200 | 200 |
| PEG 300 | 100 | 150 | 200 | 200 |
| SBECD | 500 | 450 | 400 | 350 |
| Water for Inj. | ~300 | ~300 | ~350 | ~400 |
| Observation | API soluble | API soluble, | API soluble | API soluble |
| of the | | but the | | |
| formulation | | solution | | |
| | | viscosity | | |
| | | is high. | | |
| Storage | Stratification | No API | No API | No API |
| stability | occurred | precipitation | precipitation | precipitation |
| (25° C.) | after two months | after 12 months | after 12 months | after 12 months |
| Dilution | 8 hours | 3 hours | 3 hours | 3 hours |
| stability time | | | | |
| (observation of | | | | |
| API precipitation) | | | | |
| Results | Not enough | | | |
| explanation | PEG300 in | | | |
| | the formulation, | | | |
| | cause | | | |
| | stratification | | | |

From Table 4, compositions comprising cabazitaxel showed that large quantity of ethanol did help to increase the API, cabazitaxel, solubility. In addition, the data from Table 4 indicates that quantity of PEG 300 may be critical as well. Not enough PEG 300 in the formulation may cause separation or stratification of the solution.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A liquid pharmaceutical composition comprising:
a cabazitaxel complex with a β-cyclodextrin derivative, wherein the β-cyclodextrin derivative is selected from the group consisting of sulfobutylether-β-cyclodextrin sodium, hydroxypropyl-sulfobutylether-β-cyclodextrin, and combination thereof;
an alcohol suitable for parenteral administration; and
water.

2. The liquid pharmaceutical composition of claim 1, further comprises a low molecular weight polyethylene glycol (PEG).

3. The liquid pharmaceutical composition of claim 2, further comprises povidone.

4. The liquid pharmaceutical composition of claim 3, which comprises a selected amount of the cabazitaxel by weight, about 5 to about 200 parts by weight (p.b.w.) of the β-cyclodextrin derivative relative to the cabazitaxel, about 5 to about 200 p.b.w. of the PEG relative to the cabazitaxel, about 5 to about 60 p.b.w. of the alcohol relative to the cabazitaxel, and about 10 to about 50 p.b.w. of the water relative to the cabazitaxel.

5. The liquid pharmaceutical composition of claim 2, which comprises a weight ratio of cabazitaxel to β-cyclodextrin derivative to PEG of about 1:10:30, about 1:20:30, about 1:30:20, about 1:40:20, about 1:50:10, about 1:60:30, about 1:70:20, about 1:80:20, about 1:90:10or about 1:100:10.

6. The liquid pharmaceutical composition of claim 3 which comprises a weight ratio of cabazitaxel to β-cyclodextrin derivative to PEG to alcohol to water to povidone of about 1:10:30:20:10, 1:20:30:20:10, 1:30:30:20:10, about 1:40:30:30:20, 1:50:30:20:10, about 1:60:20:5:30, about 1:70:20:40:40, about 1:80:20:40:40, or about 1:100:10:10:50.

7. The liquid pharmaceutical composition of claim 6, further comprising one or more of a weak organic acid, and an antioxidant.

8. The liquid pharmaceutical composition of claim 7, wherein the antioxidant is selected from the group consisting of sodium bisulfite, sodium metabisulfite, and combinations thereof.

9. The liquid pharmaceutical composition of claim 7, wherein the weak organic acid comprises citric acid.

10. The liquid pharmaceutical composition of claim 8 further comprises about 2-5 p.b.w. of the weak organic acid, and about 0.01-0.1 p.b.w. of the antioxidant.

11. The liquid pharmaceutical composition of claim 2, wherein the polyethylene glycol is selected from the group consisting of PEG 200, PEG 300, PEG 400, PEG 600, and combinations thereof.

12. The liquid pharmaceutical composition of claim 1, wherein the alcohol suitable for parenteral administration is selected from the group consisting of ethanol, n-propanol, isopropanol, benzyl alcohol, and combinations thereof.

13. The liquid pharmaceutical composition of claims 2, which comprises about 40 to about 200 p.b.w. of the β-cyclodextrin derivative relative to the cabazitaxel and about 10 to about 30 p.b.w. of the PEG relative to the cabazitaxel.

14. A method of making the liquid pharmaceutical composition, comprising:
a) dissolving a cabazitaxel in an alcohol suitable for parenteral administration to form a taxane solution;
b) dispersing a low molecular weight polyethylene glycol and/or PVP in water to form a dispersion, and dissolving a β-cyclodextrin derivative in the dispersion;
c) combining the cabazitaxel solution obtained in step (a) and the dispersion obtained in step (b) to obtain a homogeneous solution; and
d) as needed, adjusting the homogeneous solution obtained in step (c) to a final volume with the alcohol or water.

15. The method of claim 14, wherein the homogeneous solution is filter-sterilized.

16. The method of claim 14, wherein the alcohol comprises ethanol, the weak organic acid comprises citric acid, the antioxidant is selected from the group consisting of sodium bisulfite, sodium metabisulfite and combinations thereof.

17. A method of treating a cancer patient comprising parenterally administering the liquid pharmaceutical formulation comprising cabazitaxel thereof to the cancer patient in an amount sufficient to treat the cancer.

18. The method of claims 17, wherein the liquid pharmaceutical formulation further comprises β-cyclodextrin derivative. wherein the β-cyclodextrin derivative is selected from the group consisting of sulfobutylether-β-cyclodextrin sodium, hydroxypropyl-sulfobutylether-β-cyclodextrin, and combination thereof.

19. The method of claim 17, wherein the liquid pharmaceutical formulation further comprises povidone.

20. The method of claim 18, wherein a selected amount of the cabazitaxel by weight is about 5 to about 200 parts by weight (p.b.w.) of the β- [symbol]
